(19) United States
(12) Patent Application Publication
(10) Pub. No.: US 2024/0190758 A1
(43) Pub. Date: Jun. 13, 2024

(54) LITHIUM SILICATE GLASS CERAMIC WITH EASY MACHINABILITY

(71) Applicant: Ivoclar Vivadent AG, Schaan (LI)

(72) Inventors: Markus Rampf, Seewis Dorf (CH); Christian Ritzberger, Grabs (CH); Marc Dittmer, Feldkirch (AT)

(21) Appl. No.: 18/533,063

(22) Filed: Dec. 7, 2023

(30) Foreign Application Priority Data
Dec. 8, 2022 (EP) 22212273.1

(51) Int. Cl. C03C 10/00; A61C 13/00; A61C 13/083; C03C 4/00

(52) U.S. Cl. CPC C03C 10/00; A61C 13/0003; A61C 13/083; C03C 4/0021

(57) ABSTRACT

A lithium silicate glass ceramics having lithium disilicate as main crystal phase and having not more than 40 wt.-% of lithium disilicate crystals.

### CROSS-REFERENCE TO RELATED APPLICATIONS

This application claims priority to European Patent Application No. 22212273.1 filed on Dec. 8, 2022, the disclosure of which is incorporated herein by reference in its entirety.

### TECHNICAL FIELD

The invention relates to lithium silicate glass ceramics which are suitable especially for use in dentistry and in particular for the production of dental restorations, and to precursors for the production of this glass ceramic.

### BACKGROUND

Lithium silicate glass ceramics are generally characterized by very good mechanical properties, which is why they have been used for some time in the dental field, primarily for the fabrication of dental crowns and small dental bridges.

WO 95/32678 A2 and corresponding U.S. Pat. No. 5,702,514, which is hereby incorporated by reference, describe lithium disilicate glass ceramics which are processed into dental restorations by pressing in a viscous state. However, the use of a deformable crucible is mandatory, which makes the processing very complex.

EP 0 827 941 A1 and EP 0 916 625 A1 disclose lithium disilicate glass ceramics, which can be given the shape of the desired dental restoration by pressing or machining.

EP 1 505 041 A1 and corresponding U.S. Pat. No. 7,316,740, which is hereby incorporated by reference, and EP 1 688 398 A1 describe processes for the production of dental restorations of lithium disilicate glass ceramics. In these processes, a glass ceramic with lithium metasilicate as the main crystal phase is first produced as an intermediate stage, which can be machined, e.g. by means of a CAD/CAM process. This intermediate stage is then subjected to a further heat treatment to form the desired high-strength lithium disilicate glass ceramic.

The machining of conventional lithium disilicate glass ceramics is difficult due to their high strength and is therefore regularly accompanied by high wear of the tools used. Machining of lithium metasilicate glass ceramics is generally easier and possible with less tool wear. However, the shaped lithium metasilicate glass ceramics obtained in this way must undergo further heat treatment to convert lithium metasilicate crystals into lithium disilicate crystals and thus form dental restorations with sufficiently high strength. This is particularly problematic for the often desired providing of a patient with a dental restoration in a single treatment session (so-called chairside treatment).

There is therefore a need for lithium silicate glass ceramics that can be machined quickly and easily, that can be used as dental restorations without a further crystallization step, and that also exhibit high chemical resistance and excellent optical properties.

### SUMMARY

This problem is solved by the lithium silicate glass ceramic according to the claims. Also subject of the invention are the starting glass according to the claims, and the processes according to the claims.

### DETAILED DESCRIPTION

The lithium silicate glass ceramic according to the invention is characterized by the fact that it comprises lithium disilicate as main crystal phase and comprises no more than 40 wt.-% of lithium disilicate crystals.

Surprisingly, it has been found that the glass ceramic according to the invention shows a combination of very desirable mechanical and optical properties, as are required precisely for a restorative dental material. This glass ceramic has low strength and toughness and accordingly can be easily and in a very short time machined into the shape of even complicated dental restorations, but after such machining can be used even without further heat treatment as dental restoration with excellent mechanical properties, excellent optical properties and very good chemical stability.

The term “main crystal phase” is used to describe the crystal phase which has the highest proportion by mass of all the crystal phases present in the glass ceramic. The masses of the crystal phases are determined in particular by the Rietveld method. A suitable procedure for the quantitative analysis of the crystal phases by means of the Rietveld method is described, for example, in the dissertation by M. Dittmer “Glasses and glass ceramics in the system MgO—Al₂O₃—SiO₂ with ZrO₂ as nucleating agent”, University of Jena 2011.

Preferably, the glass ceramic according to the invention comprises no more than 35 wt.-%, preferably no more than 32 wt.-%, further preferably no more than 30 wt.-% and particularly preferably no more than 28 wt.-% of lithium disilicate crystals. Particularly preferably, the glass ceramic comprises 10 to 40 wt.-%, preferably 15 to 35 wt.-%, further preferably 20 to 32 and especially preferably 22 to 30 wt.-% of lithium disilicate crystals.

It is further preferred that in the glass ceramic according to the invention the lithium disilicate crystals have an average length in the range from 10 to 1000 nm, preferably in the range from 50 to 750 nm, particularly preferably in the range from 100 to 500 nm and most preferably in the range from 150 to 250 nm, and have an aspect ratio in the range from 1.0 to 5.0, preferably in the range from 1.25 to 3.0, particularly preferably in the range from 1.5 to 2.5 and most preferably in the range from 1.75 to 2.0. In this context, the term “average length” denotes the numerical average of the largest extension of the crystals and the term “average aspect ratio” denotes the numerical average of the quotients of the largest and smallest extension of the crystals. The measurement of the extensions of the crystals can be carried out in particular on the basis of SEM images, which are preferably taken on polished and HF vapor-etched surfaces of the glass ceramic in question, using image analysis software such as the Olympus Stream Motion software (Olympus Corporation, Tokyo, Japan).

The lithium silicate glass ceramic according to the invention comprises in particular 62.0 to 80.0, preferably 64.0 to 75.0 and particularly preferably 65.0 to 73.0 wt.-% SiO₂.

It is further preferred that the glass ceramic comprises 7.0 to 13.0, preferably 9.0 to 12.5, and particularly preferred 10.0 to 12.0 wt.-% Li₂O. It is believed that Li₂O lowers the viscosity of the glass matrix and thus promotes crystallization of the desired phases.

In another preferred embodiment, the glass ceramic comprises 2.0 to 12.0, preferably 3.0 to 10.0, and more preferably 5.0 to 9.0 wt.-% of further oxide of monovalent elements Me^{I}₂O, wherein Me^{I}₂O is selected from Na₂O, K₂O, Rb₂O, Cs₂O and mixtures thereof, and is preferably K₂O.

Particularly preferably, the glass ceramic comprises at least one and in particular all of the following further oxides of monovalent elements Me^{I}₂O in the amounts indicated:

| Component | Wt.-% |
|---|---|
| Na₂O | 0 to 5.0 |
| K₂O | 0 to 10.0 |
| Rb₂O | 0 to 8.0 |
| Cs₂O | 0 to 8.0. |

In a particularly preferred embodiment, the glass ceramic according to the invention comprises 2.0 to 10.0, preferably 3.0 to 9.0, and especially preferably 5.0 to 8.0 wt.-% K₂O.

It is also preferred that the glass ceramic comprises 3.0 to 12.0, preferably 3.0 to 10.0, more preferably 3.0 to 9.0, and most preferably 4.0 to 7.0 wt.-% Al₂O₃.

In another preferred embodiment, the glass ceramic comprises 0.5 to 10.0, preferably 1.0 to 8.0, more preferably 1.5 to 6.0, particularly preferably 1.8 to 5.0, and most preferably 2.0 to 3.0 wt.-% P₂O₅. The P₂O₅ is believed to act as a nucleating agent.

It is further preferred that the glass ceramic comprises 0 to 8.0, preferably 0.5 to 7.0, more preferably 1.0 to 6.0, and most preferably 2.0 to 4.0 wt.-% oxide of divalent elements Me^{II}O selected from the group of MgO, Cao, Sro, Zno and mixtures thereof.

In a further preferred embodiment, the glass ceramic comprises less than 4.0, preferably less than 2.0, and particularly preferably less than 1.0 wt.-% of BaO. In particular, the glass ceramic is substantially free of BaO.

Preferably, the glass ceramic comprises at least one and in particular all of the following oxides of divalent elements Me^{II}O in the amounts indicated:

| Component | Wt.-% |
|---|---|
| MgO | 0 to 4.0 |
| CaO | 0 to 4.0 |
| SrO | 0 to 10.0 |
| ZnO | 0 to 6.0. |

In a particularly preferred embodiment, the glass ceramic comprises 0 to 4.0, preferably 0.5 to 3.5, and more preferably 1.0 to 3.0 wt.-% MgO.

In another particularly preferred embodiment, the glass ceramic comprises 0 to 10.0, preferably 0.5 to 8.0, more preferably 1.0 to 6.0, and most preferably 2.0 to 4.0 wt.-% Sro.

A glass ceramic is further preferred which comprises 0 to 12.0, preferably 0.5 to 10.0, particularly preferably 1.0 to 8.0 and especially preferably 2.0 to 6.0 wt.-% of further oxide of trivalent elements Me^{III}₂O₃ selected from the group of B₂O₃, Y₂O₃, La₂O₃, Ga₂O₃, In₂O₃ and mixtures thereof.

Particularly preferably, the glass ceramic comprises at least one and in particular all of the following further oxides of trivalent elements Me^{III}₂O₃ in the amounts indicated:

| Component | Wt.-% |
|---|---|
| B₂O₃ | 0 to 8.0 |
| Y₂O₃ | 0 to 8.0 |
| La₂O₃ | 0 to 8.0 |
| Ga₂O₃ | 0 to 5.0 |
| In₂O₃ | 0 to 5.0. |

Furthermore, a glass ceramic is preferred which comprises 0 to 12.0, preferably 0.5 to 10.0, and particularly preferably 1.0 to 9.0 wt.-% oxide of tetravalent elements Me^{IV}O₂ selected from the group of TiO₂, ZrO₂, GeO₂, SnO₂, CeO₂ and mixtures thereof.

Particularly preferably, the glass ceramic comprises at least one and in particular all of the following oxides of tetravalent elements Me^{IV}O₂ in the amounts indicated:

| Component | Wt.-% |
|---|---|
| TiO₂ | 0 to 5.0 |
| ZrO₂ | 0 to 12.0 |
| GeO₂ | 0 to 5.0 |
| SnO₂ | 0 to 5.0 |
| CeO₂ | 0 to 5.0 |

In a particularly preferred embodiment, the glass ceramic comprises 0 to 12.0 preferably 1.0 to 10.0 and more preferably 4.0 to 8.0 wt.-% Zro₂.

In another embodiment, the glass ceramic comprises 0 to 10.0, preferably 1.0 to 9.0, and more preferably 2.0 to 8.0 wt.-% of further oxide of pentavalent elements Me^{V}₂O₅ selected from the group of V₂O₅, Nb₂O₅, Ta₂O₅ and mixtures thereof.

Particularly preferably, the glass ceramic comprises at least one and in particular all of the following further oxides of pentavalent elements Me^{V}₂O₅ in the amounts indicated:

| Component | Wt.-% |
|---|---|
| V₂O₅ | 0 to 2.0 |
| Nb₂O₅ | 0 to 10.0 |
| Ta₂O₅ | 0 to 10.0 |

In another embodiment, the glass ceramic comprises 0 to 8.0, preferably 1.0 to 6.0, and more preferably 2.0 to 4.0 wt.-% oxide of hexavalent elements Me^{VI}O₃ selected from the group of MoO₃, WO₃ and mixtures thereof.

Particularly preferably, the glass ceramic comprises at least one and in particular all of the following oxides Me^{VI}O₃ in the amounts indicated:

| Component | Wt.-% |
|---|---|
| MoO₃ | 0 to 3.0 |
| WO₃ | 0 to 3.0. |

In a further embodiment, the glass ceramic according to the invention comprises 0 to 5.0, preferably 0.1 to 2.0 and particularly preferably 0.5 to 1.0 wt.-% fluorine.

Particularly preferred is a glass ceramic which comprises at least one and preferably all of the following components in the amounts indicated:

| Component | Wt.-% |
|---|---|
| SiO₂ | 62.0 to 80.0 |
| Li₂O | 7.0 to 13.0 |
| Me^{I}₂O | 2.0 to 12.0, in particular 5.0 to 10.0 |
| Al₂O₃ | 3.0 to 12.0 |
| P₂O₅ | 0.5 to 10.0 |
| Me^{II}O | 0 to 8.0 |
| Me^{III}₂O₃ | 0 to |
| Me^{IV}O₂ | 0 to 12.0 |
| Me^{V}2O₅ | 0 to 10.0 |
| Me^{VI}O₃ | 0 to 8.0 |
| Fluorine | 0 to 5.0, |

wherein Me^{I}₂O, Me^{II}O, Me^{III}₂O₃, Me^{IV}O₂, Me^{V}₂O₅ and Me^{VI}O₃ have the meanings given above.

In another particularly preferred embodiment, the glass ceramic comprises at least one and preferably all, of the following components in the amounts indicated:

| Component | Wt.-% |
|---|---|
| SiO₂ | 62.0 to 80.0 |
| Li₂O | 7.0 to 13.0 |
| Al₂O₃ | 3.0 to 12.0 |
| P₂O₅ | 0.5 to 10.0 |
| Na₂O | 0 to 5.0 |
| K₂O | 0 to 10.0 |
| Rb₂O | 0 to 8.0 |
| Cs₂O | 0 to 8.0 |
| MgO | 0 to 4.0 |
| CaO | 0 to 4.0 |
| SrO | 0 to 10.0 |
| ZnO | 0 to 6.0 |
| B₂O₃ | 0 to 8.0 |
| Y₂O₃ | 0 to 8.0 |
| La₂O₃ | 0 to 8.0 |
| Ga₂O₃ | 0 to 5.0 |
| In₂O₃ | 0 to 5.0 |
| TiO₂ | 0 to 5.0 |
| ZrO₂ | 0 to 12.0 |
| GeO₂ | 0 to 5.0 |
| SnO₂ | 0 to 5.0 |
| CeO₂ | 0 to 5.0 |
| V₂O₅ | 0 to 2.0 |
| Nb₂O₅ | 0 to 10.0 |
| Ta₂O₅ | 0 to 10.0 |
| MoO₃ | 0 to 3.0 |
| WO₃ | 0 to 3.0 |
| Fluorine | 0 to 5.0. |

Some of the above components may serve as colorants and/or fluorescent agents. The glass ceramic according to the invention may furthermore contain further colorants and/or fluorescent agents. These may be selected, for example, from Bi₂O₃ or Bi₂O₅ and in particular from further inorganic pigments and/or oxides of d and f elements, such as the oxides of Mn, Fe, Co, Pr, Nd, Tb, Er, Dy, Eu and Yb. By means of these colorants and fluorescent agents, it is possible to easily color the glass ceramic to imitate the desired optical properties, in particular of natural dental material.

In a preferred embodiment of the glass ceramic, the molar ratio of SiO₂ to Li₂O is in the range of 2.5 to 4.0, preferably in the range of 2.8 to 3.8, and particularly preferably in the range of 3.0 to 3.6.

Typically, the glass ceramic according to the invention comprises, in addition to the main crystal phase lithium disilicate, at most small amounts of secondary crystal phases. Preferably, the glass ceramic comprises less than 12 wt.-%, in particular less than 10 wt.-%, preferably less than 8 wt.-% and particularly preferably less than 5 wt.-% of secondary crystal phases. Examples of such secondary crystal phases are lithium metasilicate crystals, lithium phosphate crystals, SiO₂ crystals such as quartz crystals or cristobalite crystals, SiO₂ solid solutions such as quartz solid solutions, cristobalite solid solutions or lithium aluminosilicate crystals, or ZrO₂ crystals. It is particularly preferred that the glass ceramic comprises less than 10 wt.-%, in particular less than 7 wt.-%, preferably less than 5 wt.-%, especially preferably less than 3 wt.-% and most preferably less than 1 wt.-% of quartz crystals and/or quartz solid solutions. It is also particularly preferred that the glass ceramic according to the invention is essentially free of cristobalite.

The type and, in particular, the amount of crystal phases formed can be controlled by the composition of the starting glass as well as the heat treatment applied to produce the glass ceramic from the starting glass. The examples illustrate this by varying the composition of the starting glass and the heat treatment applied.

The glass ceramic has a biaxial fracture strength OB of preferably at least 200 MPa and particularly preferably 250 to 600 MPa. The biaxial fracture strength was determined in accordance with ISO 6872 (2008) (piston-on-three-balls test).

The glass ceramic according to the invention has a thermal expansion coefficient CTE (measured in the range from 100 to 500° C.) of preferably 8 to 13·10⁻⁶ K⁻¹. The CTE is determined according to ISO 6872 (2015). The coefficient of thermal expansion is adjusted to a desired value in particular by the type and quantity of crystal phases present in the glass ceramic and the chemical composition of the glass ceramic.

The translucency of the glass ceramic was determined in terms of the contrast value (CR value) according to British Standard BS 5612, and this contrast value was preferably 30 to 90 and more preferably 40 to 85.

The invention also relates to various precursors with corresponding composition from which the lithium silicate glass ceramic according to the invention can be produced by heat treatment. These precursors are a correspondingly composed starting glass and a correspondingly composed starting glass with nuclei. The term “corresponding composition” means that these precursors comprise the same components in the same amounts as the glass ceramic, the components being calculated as oxides with the exception of fluorine, as is usual for glasses and glass ceramics.

The invention therefore also relates to a starting glass comprising the components of the lithium silicate glass ceramic according to the invention.

The starting glass according to the invention therefore comprises, in particular, suitable amounts of SiO₂ and Li₂O, which are required to form the glass ceramic according to the invention with lithium disilicate as main crystal phase. Further, the starting glass may also comprise other components as indicated above for the lithium silicate glass ceramic according to the invention. All such embodiments are preferred for the components of the starting glass that are also indicated as preferred for the components of the lithium silicate glass ceramic according to the invention.

The invention also relates to such a starting glass comprising nuclei for the formation of lithium disilicate crystals.

The lithium silicate glass ceramics according to the invention and the starting glass according to the invention are present in particular in the form of powders, granules or blanks of any shape and size, e.g. monolithic blanks, such as platelets, cuboids or cylinders, or powder compacts, in unsintered, partially sintered or densely sintered form. In these forms, they can be easily further processed. However, they can also be in the form of dental restorations, such as inlays, onlays, crowns, veneers, facets or abutments.

In particular, the starting glass is produced by melting a mixture of suitable starting materials, such as carbonates, oxides, phosphates and fluorides, at temperatures of, in particular, 1300 to 1600° C. for 2 to 10 hours. To achieve a particularly high homogeneity, the glass melt obtained is poured into water to form a glass granulate, and the granulate obtained is then remelted.

The melt can then be poured into molds to produce blanks of the starting glass, so-called solid glass blanks or monolithic blanks.

It is also possible to pour the melt into water again to produce a granulate. After grinding and, if necessary, adding further components, such as coloring and fluorescent agents, this granulate can be pressed into a blank, a so-called powder compact.

Finally, the starting glass can also be processed into a powder after granulation.

Subsequently, the starting glass, e.g. in the form of a solid glass blank, a powder compact or in the form of a powder, is subjected to at least one heat treatment. It is preferred that first of all a first heat treatment is carried out to produce the starting glass according to the invention with nuclei for the formation of lithium disilicate crystals. The starting glass with nuclei is then typically subjected to at least one further heat treatment at a higher temperature to effect crystallization of lithium disilicate and to produce the lithium silicate glass ceramic according to the invention.

The invention thus also relates to a process for the production of the lithium silicate glass ceramic according to the invention, in which the starting glass or the starting glass with nuclei is subjected to at least one heat treatment at a temperature of 400 to 1000° C. for a duration of, in particular, 1 to 120 min, preferably 5 to 90 min and particularly preferably 10 to 60 min.

The at least one heat treatment carried out in the process according to the invention can also be carried out in the course of hot pressing or sintering of the starting glass according to the invention or the starting glass according to the invention with nuclei.

It is preferred to subject the starting glass to a heat treatment at a temperature of 400 to 650° C., preferably 450 to 600° C. and particularly preferably 480 to 580° C., for a duration of in particular 1 to 240 min, preferably 5 to 120 min and particularly preferably 10 to 60 min, in order to produce the starting glass with nuclei for the crystallization of lithium disilicate.

It is further preferred to subject the starting glass with nuclei to a heat treatment at a temperature of 700 to 1000° C., preferably 750 to 950° C. and particularly preferably 800 to 900° C., for a duration of in particular 1 to 120 min, preferably 2 to 90 min, particularly preferably 5 to 60 min and most preferably 10 to 30 min, in order to produce the lithium silicate glass ceramic. The suitable conditions for a given glass ceramic can be determined, for example, by performing X-ray diffraction analyses at different temperatures.

In a preferred embodiment, the process for producing the lithium silicate glass ceramic according to the invention thus comprises that
(a) the starting glass is subjected to a heat treatment at a temperature of 400 to 650° C., preferably 450 to 600° C. and more preferably 480 to 580° C., for a duration of in particular 1 to 240 min, preferably 5 to 120 min and more preferably 10 to 60 min, in order to form starting glass with nuclei, and
(b) the starting glass with nuclei is subjected to a heat treatment at a temperature of 700 to 1000° C., preferably 750 to 950° C. and particularly preferably 800 to 900° C., for a duration of in particular 1 to 120 min, preferably 2 to 90 min, particularly preferably 5 to 60 min and most preferably 10 to 30 min, to form the glass ceramic.

Due to the above-described properties of the glass ceramics according to the invention and the glasses according to the invention, they are particularly suitable for use in dentistry. The invention therefore also relates to the use of the glass ceramics according to the invention or the glasses according to the invention as dental material and in particular for the production of dental restorations or as coating material for dental restorations.

In particular, the glass ceramics according to the invention and the glasses according to the invention can be used to produce dental restorations, such as bridges, inlays, onlays, veneers, abutments, partial crowns, crowns or facets. The invention therefore also relates to the use of the glass ceramics according to the invention or the glasses according to the invention for the production of dental restorations. In this context, it is preferred that the glass ceramic or the glass is given the shape of the desired dental restoration by pressing or machining.

The invention also relates to a process for producing dental restorations, in which the glass ceramics or glasses according to the invention are given the shape of the desired dental restoration by pressing or machining.

The pressing is usually carried out under elevated pressure and at elevated temperature. It is preferred that the pressing is carried out at a temperature of 700 to 1200° C. It is further preferred that the pressing is carried out at a pressure of 2 to 10 bar. During pressing, the desired change in shape is achieved by viscous flow of the material used. The starting glass according to the invention and, in particular, the starting glass with nuclei according to the invention, and the lithium silicate glass ceramic according to the invention can be used for the pressing. The glasses and glass ceramics according to the invention can be used in particular in the form of blanks of any shape and size, e.g. solid blanks or powder compacts, e.g. in unsintered, partially sintered or densely sintered form.

Machining is usually performed by material removing processes and in particular by milling and/or grinding. It is particularly preferred that the machining is carried out as part of a CAD/CAM process. The starting glass according to the invention, the starting glass with nuclei according to the invention and the lithium silicate glass ceramic according to the invention can be used for the machining. In this context, the glasses and glass ceramics according to the invention can be used in particular in the form of blanks, e.g. solid blanks or powder compacts, e.g. in unsintered, partially sintered or densely sintered form. The lithium silicate glass ceramic according to the invention is preferably used for machining.

Surprisingly, it has been shown that the lithium silicate glass ceramics according to the invention can be machined faster than known lithium silicate glass ceramics when the same force is applied. To describe this property, in particular the removal rate on sample bodies of the glass ceramics can be determined. For this purpose, platelets are sawn off the specimens and weighed. The platelets are then glued to a holder and ground under water cooling with an automatic grinding machine, such as those available from Struers, using a diamond grinding wheel, for example with a grain size of 20 μm. The pressure of the grinding machine is selected so that the same force, for example 15 N, is applied to each platelet. After the platelets have been ground for 1 min, they are dried and weighed again. The removal rate is then calculated according to the following formula:

Removal rate [wt.-%·min⁻¹]=100×(1−(mground:munground))

After the glass ceramic has obtained the shape of the desired dental restoration, it can be subjected to further heat treatment to bring about further growth of lithium disilicate crystals. For example, for this purpose the glass ceramic is subjected to a heat treatment at a temperature of 700 to 1000° C., preferably 750 to 950° C. and particularly preferably 800 to 900° C., in particular for a duration of 1 to 90 minutes, preferably 2 to 60 minutes, more preferably 5 to 30 minutes and most preferably 10 to 15 minutes.

However, it has been surprisingly shown that the machinable lithium silicate glass ceramic with lithium disilicate as main crystal phase, even without further heat treatment, not only exhibits mechanical properties, such as sufficient strength, but also shows other properties required for a material for dental restorations. Therefore, the glass ceramic according to the invention is used as a dental material preferably without further heat treatment.

However, the glass ceramics according to the invention and the glasses according to the invention are also suitable as coating material of, for example, ceramics and glass ceramics. The invention is therefore also directed to the use of the glasses according to the invention or the glass ceramics according to the invention for coating ceramics, glass ceramics and in particular dental restorations.

The invention also relates to a process for coating ceramics, metals, metal alloys and glass ceramics, in which glass ceramic or glass according to the invention is applied to the ceramic or glass ceramic and subjected to elevated temperature.

This can be done in particular by sintering on or by joining an overlay produced by CAD-CAM with a suitable glass solder or adhesive and preferably by pressing on. In the case of sintering on, the glass ceramic or glass is applied in the usual manner, e.g. as a powder, to the material to be coated, such as ceramic or glass ceramic, and then sintered at elevated temperature. In the preferred pressing-on process, the glass ceramic or glass according to the invention, for example in the form of powder compacts or monolithic blanks, is pressed on at an elevated temperature, of, for example, 700 to 1200° C., and with the application of pressure, for example 2 to 10 bar. In particular, the processes described in EP 231 773 and the pressing furnace disclosed therein can be used for this purpose. A suitable furnace is, for example, the Programat EP 5000 from Ivoclar Vivadent AG, Liechtenstein.

It is preferred that, after completion of the coating process, a glass ceramic with lithium silicate, in particular lithium disilicate, is present as main crystal phase, since such a glass ceramic has particularly good properties.

The invention is explained in more detail below by means of examples which do not limit it.

### Examples

A total of 77 glasses according to the invention and one comparative example with the compositions given in Table I were produced. The glasses were crystallized into glass ceramics according to Table II. The following meanings apply
T_{g}: Glass transition temperature determined by DSC
T_{S} and t_{S}: Applied temperature and time for melting
T₁ and t₁: Applied temperature and time for 1st heat treatment
T₂ and t₂: Applied temperature and time for 2nd heat treatment

First, starting glasses with the compositions given in Table I were melted on a 100 to 200 g scale from common raw materials at the temperature T_{S} for the duration t_{S}, whith the melting being very well possible without the formation of bubbles or streaks. Glass granules were produced by pouring these starting glasses into water.

In Examples 1-70, the glass granules were melted a second time at temperature T_{S} for duration t_{S} for homogenization. The obtained melts of the starting glass were poured into a graphite mold to produce solid glass blocks. A first heat treatment of the obtained glass monoliths at the temperature T₁ for the duration t₁ resulted in the formation of glasses with nuclei. These nucleated glasses crystallized into glass ceramics by further heat treatment at temperature T₂ for duration t₂.

In Examples 71-77, the glass granules were dried in a drying furnace at 150° C. for 1 h, ground to <90 μm in a mill lined with zirconium oxide and sieved. The resulting glass powders were pressed into cuboid blanks using a uniaxial press at 10 bar. The blanks were first heated under vacuum at a heating rate of 10 K/min to the temperature T₁ and held at this temperature for the duration t₁. Subsequently, the blanks were further heated to the temperature T₂ at a heating rate of 10 K/min and held at this temperature for the duration t₂. Finally, the blanks were cooled to room temperature.

As determined by X-ray diffraction studies at room temperature, glass ceramics with lithium disilicate as main crystal phase were obtained in all cases.

The amounts of the crystal phases were determined by means of X-ray diffraction. For this purpose, a powder of the respective glass ceramic was prepared by grinding and sieving (<45 μm) and mixed with Al₂O₃ (Alfa Aesar, Product No. 42571) as internal standard in a ratio of 80 wt.-% glass ceramic to 20 wt.-% Al₂O₃. This mixture was slurried with acetone to achieve the best possible mixing. The mixture was then dried at about 80° C. A diffractogram was then recorded in the range 10 to 100° 2θ using a D8 Advance diffractometer from Bruker using CuKα radiation and a step size of 0.014° 2θ. This diffractogram was then evaluated using Bruker's TOPAS 5.0 software using the Rietveld method.

The average length and the average aspect ratio of the lithium disilicate crystals were determined from SEM images. For this purpose, a surface of the respective glass ceramic was polished (<0.5 μm), etched with 40% HF vapor for at least 30 s and then sputtered with an Au—Pd layer. SEM images were taken of the surfaces treated in this way using a Supra 40VP scanning electron microscope (Zeiss, Oberkochen, Germany). The SEM images were post-processed to improve the contrast between crystals and glass phase using a common image processing program. Subsequently, the mean length and mean aspect ratio of the crystals were determined using Olympus Stream Motion 2.4 image analysis software (Olympus Corporation, Tokyo, Japan).

To determine the machinability, two platelets each with an area of 170 mm²±10 mm²(about 12.5 mm×13.8 mm) and a thickness of 4.0±0.5 mm were sawn off the glass ceramic blocks obtained in this way and weighed on a precision balance. The platelets were then glued onto a holder and ground under water cooling with an automatic grinding machine (LaboForche-100, Struers) using a diamond grinding wheel with a grain size of 20 μm. The pressure of the grinding machine was selected so that a force of 15 N was applied to each platelet. The turntable, on which the diamond grinding wheel was mounted, and the head of the grinding machine, on which the holder with the specimens was mounted, had the same direction of rotation. The speed of the turntable was 300 rpm⁻¹. The platelets were ground for 1 min and then dried and reweighed. The removal rate was calculated according to the following formula:

Removal rate [wt.-%·min⁻¹]=100×(1−(mground:munground))

As can be seen from Table II, the removal rates for the examples according to the invention were consistently higher than in the comparative example. This shows that the lithium silicate glass ceramics according to the invention can be machined faster than known lithium disilicate glass ceramics when applying the same force.

TABLE I

| | Example | | | | | | | | | |
|---|---|---|---|---|---|---|---|---|---|---|
| | 1 | 2 | 3 | 4 | 5 | 6 | 7 | 8 | 9 | 10 |
| Composition | Wt.-% | Wt.-% | Wt.-% | Wt.-% | Wt.-% | Wt.-% | Wt.-% | Wt.-% | Wt.-% | Wt.-% |
| SiO₂ | 68.8 | 67.8 | 67.0 | 67.6 | 66.4 | 66.6 | 64.2 | 69.7 | 70.1 | 66.2 |
| Li₂O | 9.9 | 9.7 | 9.5 | 9.6 | 9.4 | 9.5 | 9.1 | 11.5 | 11.7 | 11.0 |
| Na₂O | — | — | — | — | — | — | — | — | — | 3.0 |
| K₂O | 5.8 | 5.7 | 5.6 | 5.7 | 5.5 | 5.6 | 5.4 | 3.3 | 2.3 | — |
| Rb₂O | — | — | — | — | — | — | — | — | — | 5.0 |
| Cs₂O | — | — | — | — | — | — | — | — | — | — |
| MgO | — | 1.4 | 2.7 | — | — | — | — | — | — | — |
| CaO | — | — | — | 1.9 | 3.8 | 3.4 | — | — | — | — |
| SrO | — | — | — | — | — | — | 6.7 | — | — | — |
| ZnO | — | — | — | — | — | — | — | — | — | — |
| Al₂O₃ | 5.0 | 5.0 | 4.9 | 4.9 | 4.8 | 4.8 | 4.7 | 4.6 | 4.7 | 4.0 |
| Y₂O₃ | — | — | — | — | — | — | — | 2.0 | 2.1 | — |
| La₂O₃ | — | — | — | — | — | — | — | | | — |
| ZrO₂ | 8.0 | 7.9 | 7.8 | 7.8 | 7.7 | 7.7 | 7.5 | 6.1 | 6.2 | 8.0 |
| CeO₂ | — | — | — | — | — | — | — | — | — | — |
| P₂O₅ | 2.5 | 2.5 | 2.5 | 2.5 | 2.4 | 2.4 | 2.4 | 2.8 | 2.9 | 2.8 |
| V₂O₅ | — | — | — | — | — | — | — | — | — | — |
| Nb₂O₅ | — | — | — | — | — | — | — | — | — | — |
| Ta₂O₅ | — | — | — | — | — | — | — | — | — | — |
| Gd₂O₃ | — | — | — | — | — | — | — | — | — | — |
| Tb₄O₇ | — | — | — | — | — | — | — | — | — | — |
| Er₂O₃ | — | — | — | — | — | — | — | — | — | — |
| F | — | — | — | — | — | — | — | — | — | — |
| Σ | 100.0 | 100.0 | 100.0 | 100.0 | 100.0 | 100.0 | 100.0 | 100.0 | 100.0 | 100.0 |
| | Example | | | | | | | | | |
| | 11 | 12 | 13 | 14 | 15 | 16 | 17 | 18 | 19 | 20 |
| Composition | Wt.-% | Wt.-% | Wt.-% | Wt.-% | Wt.-% | Wt.-% | Wt.-% | Wt.-% | Wt.-% | Wt.-% |
| SiO₂ | 65.4 | 68.8 | 68.8 | 68.7 | 68.9 | 68.9 | 68.9 | 68.9 | 67.5 | 67.7 |
| Li₂O | 10.8 | 11.4 | 9.8 | 9.8 | 9.8 | 9.8 | 9.8 | 9.8 | 11.2 | 11.2 |
| Na₂O | 3.00 | 2.0 | — | — | — | — | — | — | — | — |
| K₂O | — | 3.0 | 5.8 | 5.8 | 5.8 | 5.8 | 5.8 | 5.8 | 5.8 | 5.8 |
| Rb₂O | — | — | — | — | — | — | — | — | — | — |
| Cs₂O | 6.0 | — | — | — | — | — | — | — | — | — |
| MgO | — | — | — | — | — | — | — | — | — | — |
| CaO | — | — | — | — | — | — | — | — | — | — |
| SrO | — | — | — | — | 2.0 | 4.0 | — | — | — | — |
| ZnO | — | — | — | — | — | — | — | — | — | — |
| Al₂O₃ | 4.0 | 4.0 | 5.0 | 5.0 | 5.0 | 5.0 | 5.0 | 5.0 | 5.0 | 4.5 |
| Y₂O₃ | — | — | — | — | — | — | — | — | — | — |
| La₂O₃ | — | — | — | — | — | — | — | — | — | — |
| ZrO₂ | 8.0 | 8.0 | 8.0 | 8.0 | 6.0 | 4.0 | 6.0 | 4.0 | 8.0 | 8.0 |
| CeO₂ | — | — | — | — | — | — | — | — | — | — |
| P₂O₅ | 2.8 | 2.8 | 2.6 | 2.7 | 2.5 | 2.5 | 2.5 | 2.5 | 2.5 | 2.8 |
| V₂O₅ | — | — | — | — | — | — | — | — | — | — |
| Nb₂O₅ | — | — | — | — | — | — | — | — | — | — |
| Ta₂O₅ | — | — | — | — | — | — | 2.0 | 4.0 | — | — |
| Gd₂O₃ | — | — | — | — | — | — | — | — | — | — |
| Tb₄O₇ | — | — | — | — | — | — | — | — | — | — |
| Er₂O₃ | — | — | — | — | — | — | — | — | — | — |
| F | — | — | — | — | — | — | — | — | — | — |
| Σ | 100.0 | 100.0 | 100.0 | 100.0 | 100.0 | 100.0 | 100.0 | 100.0 | 100.0 | 100.0 |
| | Example | | | | | | | | | |
| | 21 | 22 | 23 | 24 | 25 | 26 | 27 | 28 | 29 | 30 |
| Composition | Wt.-% | Wt.-% | Wt.-% | Wt.-% | Wt.-% | Wt.-% | Wt.-% | Wt.-% | Wt.-% | Wt.-% |
| SiO₂ | 68.1 | 68.1 | 68.1 | 68.1 | 68.4 | 68.5 | 68.8 | 69.4 | 69.0 | 69.2 |
| Li₂O | 11.3 | 11.3 | 11.3 | 11.3 | 11.3 | 11.4 | 11.4 | 10.8 | 10.7 | 10.7 |
| Na₂O | — | — | — | — | — | — | — | — | — | — |
| K₂O | 5.8 | 5.8 | 5.8 | 5.8 | 5.5 | 5.3 | 5.0 | 5.0 | 5.5 | 5.5 |
| Rb₂O | — | — | — | — | — | — | — | — | — | — |
| Cs₂O | — | — | — | — | — | — | — | — | — | — |
| MgO | — | — | — | — | — | — | — | — | — | — |
| CaO | — | — | — | — | — | — | — | — | — | — |
| SrO | — | — | — | — | — | — | — | — | — | — |
| ZnO | — | — | — | — | — | — | — | — | — | — |
| Al₂O₃ | 4.0 | 4.0 | 4.0 | 4.0 | 4.0 | 4.0 | 4.0 | 4.0 | 4.0 | 4.0 |
| Y₂O₃ | — | — | — | — | — | — | — | — | — | — |
| La₂O₃ | — | 0.5 | 1.0 | — | — | — | — | — | — | — |
| ZrO₂ | 8.0 | 7.5 | 7.0 | 8.0 | 8.0 | 8.0 | 8.0 | 8.0 | 8.0 | 8.0 |
| CeO₂ | — | — | — | — | — | — | — | — | — | — |
| P₂O₅ | 2.8 | 2.8 | 2.8 | 2.8 | 2.8 | 2.8 | 2.8 | 2.8 | 2.8 | 2.6 |
| V₂O₅ | — | — | — | — | — | — | — | — | — | — |
| Nb₂O₅ | — | — | — | — | — | — | — | — | — | — |
| Ta₂O₅ | — | — | — | — | — | — | — | — | — | — |
| Gd₂O₃ | — | — | — | — | — | — | — | — | — | — |
| Tb₄O₇ | — | — | — | — | — | — | — | — | — | — |
| Er₂O₃ | — | — | — | — | — | — | — | — | — | — |
| F | — | — | — | — | — | — | — | — | — | — |
| Σ | 100.0 | 100.0 | 100.0 | 100.0 | 100.0 | 100.0 | 100.0 | 100.0 | 100.0 | 100.0 |
| | Example | | | | | | | | | |
| | 31 | 32 | 33 | 34 | 35 | 36 | 37 | 38 | 39 | 40 |
| Composition | Wt.-% | Wt.-% | Wt.-% | Wt.-% | Wt.-% | Wt.-% | Wt.-% | Wt.-% | Wt.-% | Wt.-% |
| SiO₂ | 69.3 | 70.0 | 69.8 | 70.0 | 70.3 | 70.5 | 70.3 | 70.6 | 71.1 | 70.8 |
| Li₂O | 10.8 | 10.2 | 10.2 | 10.3 | 10.3 | 9.7 | 9.7 | 9.8 | 9.8 | 10.4 |
| Na₂O | — | — | — | — | — | — | — | — | — | — |
| K₂O | 5.5 | 5.0 | 5.2 | 5.2 | 5.2 | 5.0 | 5.2 | 5.2 | 5.2 | 5.0 |
| Rb₂O | — | — | — | — | — | — | — | — | — | — |
| Cs₂O | — | — | — | — | — | — | — | — | — | — |
| MgO | — | — | — | — | — | — | — | — | — | — |
| CaO | — | — | — | — | — | — | — | — | — | — |
| SrO | — | — | — | — | — | — | — | — | — | — |
| ZnO | — | — | — | — | — | — | — | — | — | — |
| Al₂O₃ | 4.0 | 4.0 | 4.0 | 4.0 | 4.0 | 4.0 | 4.0 | 4.0 | 4.0 | 4.0 |
| Y₂O₃ | — | — | — | — | — | — | — | — | — | — |
| La₂O₃ | — | — | — | — | — | — | — | — | — | — |
| ZrO₂ | 8.0 | 8.0 | 8.0 | 8.0 | 8.0 | 8.0 | 8.0 | 8.0 | 8.0 | 7.0 |
| CeO₂ | — | — | — | — | — | — | — | — | — | — |
| P₂O₅ | 2.4 | 2.8 | 2.8 | 2.5 | 2.2 | 2.8 | 2.8 | 2.4 | 1.9 | 2.8 |
| V₂O₅ | — | — | — | — | — | — | — | — | — | — |
| Nb₂O₅ | — | — | — | — | — | — | — | — | — | — |
| Ta₂O₅ | — | — | — | — | — | — | — | — | — | — |
| Gd₂O₃ | — | — | — | — | — | — | — | — | — | — |
| Tb₄O₇ | — | — | — | — | — | — | — | — | — | — |
| Er₂O₃ | — | — | — | — | — | — | — | — | — | — |
| F | — | — | — | — | — | — | — | — | — | — |
| Σ | 100.0 | 100.0 | 100.0 | 100.0 | 100.0 | 100.0 | 100.0 | 100.0 | 100.0 | 100.0 |
| | Example | | | | | | | | | |
| | 41 | 42 | 43 | 44 | 45 | 46 | 47 | 48 | 49 | 50 |
| Composition | Wt.-% | Wt.-% | Wt.-% | Wt.-% | Wt.-% | Wt.-% | Wt.-% | Wt.-% | Wt.-% | Wt.-% |
| SiO₂ | 71.7 | 71.3 | 72.6 | 68.1 | 70.0 | 70.5 | 68.1 | 68.1 | 68.1 | 68.9 |
| Li₂O | 10.5 | 10.4 | 10.6 | 11.3 | 10.2 | 9.7 | 11.3 | 11.3 | 11.3 | 9.8 |
| Na₂O | — | — | — | — | — | — | — | — | — | — |
| K₂O | 5.0 | 5.5 | 5.0 | 5.8 | 5.0 | 5.0 | 5.1 | 5.1 | 5.1 | 5.8 |
| Rb₂O | — | — | — | — | — | — | — | — | — | — |
| Cs₂O | — | — | — | — | — | — | — | — | — | — |
| MgO | — | — | — | — | — | — | — | — | — | — |
| CaO | — | — | — | — | — | — | — | — | 0.7 | — |
| SrO | — | — | — | — | — | — | 0.7 | 0.7 | — | — |
| ZnO | — | — | — | — | — | — | — | — | — | — |
| Al₂O₃ | 4.0 | 4.0 | 4.0 | 4.0 | 4.0 | 4.0 | 4.0 | 4.0 | 4.0 | 5.0 |
| Y₂O₃ | — | — | — | 0.5 | 1.0 | 2.0 | 1.0 | — | — | — |
| La₂O₃ | — | — | — | — | — | — | — | 1.0 | 1.0 | — |
| ZrO₂ | 6.0 | 6.0 | 5.0 | 7.5 | 7.0 | 6.0 | 7.0 | 7.0 | 7.0 | — |
| CeO₂ | — | — | — | — | — | — | — | — | — | — |
| P₂O₅ | 2.8 | 2.8 | 2.8 | 2.8 | 2.8 | 2.8 | 2.8 | 2.8 | 2.8 | 2.5 |
| V₂O₅ | — | — | — | — | — | — | — | — | — | — |
| Nb₂O₅ | — | — | — | — | — | — | — | — | — | 8.0 |
| Ta₂O₅ | — | — | — | — | — | — | — | — | — | — |
| Gd₂O₃ | — | — | — | — | — | — | — | — | — | — |
| Tb₄O₇ | — | — | — | — | — | — | — | — | — | — |
| Er₂O₃ | — | — | — | — | — | — | — | — | — | — |
| F | — | — | — | — | — | — | — | — | — | — |
| Σ | 100.0 | 100.0 | 100.0 | 100.0 | 100.0 | 100.0 | 100.0 | 100.0 | 100.0 | 100.0 |
| | Example | | | | | | | | | |
| | 51 | 52 | 53 | 54 | 55 | 56 | 57 | 58 | 59 | 60 |
| Composition | Wt.-% | Wt.-% | Wt.-% | Wt.-% | Wt.-% | Wt.-% | Wt.-% | Wt.-% | Wt.-% | Wt.-% |
| SiO₂ | 68.9 | 72.4 | 70.7 | 70.7 | 71.4 | 73.5 | 71.8 | 71.8 | 72.5 | 72.4 |
| Li₂O | 9.8 | 10.3 | 10.0 | 10.0 | 10.1 | 10.5 | 10.2 | 10.2 | 10.3 | 10.3 |
| Na₂O | — | — | — | — | — | — | — | — | — | — |
| K₂O | 5.8 | 5.8 | 5.8 | 5.8 | 5.0 | 5.8 | 5.8 | 5.8 | 5.0 | 5.8 |
| Rb₂O | — | — | — | — | — | — | — | — | — | — |
| Cs₂O | — | — | — | — | — | — | — | — | — | — |
| MgO | — | — | — | — | — | — | — | — | — | — |
| CaO | — | — | — | — | — | — | — | — | — | — |
| SrO | — | — | — | — | — | — | — | — | — | — |
| ZnO | — | — | — | — | — | — | — | — | — | — |
| Al₂O₃ | 5.0 | 5.0 | 5.0 | 5.0 | 5.0 | 5.0 | 5.0 | 5.0 | 5.0 | 5.0 |
| Y₂O₃ | — | — | — | — | — | 2.7 | 2.7 | 2.7 | 2.7 | — |
| La₂O₃ | — | 4.0 | 4.0 | 4.0 | 4.0 | — | — | — | — | — |
| ZrO₂ | — | — | — | — | — | — | — | — | — | — |
| CeO₂ | — | — | — | — | — | — | — | — | — | — |
| P₂O₅ | 2.5 | 2.5 | 2.5 | 2.5 | 2.5 | 2.5 | 2.5 | 2.5 | 2.5 | 2.5 |
| V₂O₅ | — | — | — | — | — | — | — | — | — | — |
| Nb₂O₅ | — | — | — | 2.0 | 2.0 | — | — | 2.0 | — | — |
| Ta₂O₅ | 8.0 | — | 2.0 | — | — | — | 2.0 | — | 2.0 | — |
| Gd₂O₃ | — | — | — | — | — | — | — | — | — | 4.0 |
| Tb₄O₇ | — | — | — | — | — | — | — | — | — | — |
| Er₂O₃ | — | — | — | — | — | — | — | — | — | — |
| F | — | — | — | — | — | — | — | — | — | — |
| Σ | 100.0 | 100.0 | 100.0 | 100.0 | 100.0 | 100.0 | 100.0 | 100.0 | 100.0 | 100.0 |
| | Example | | | | | | | | | |
| | 61 | 62 | 63 | 64 | 65 | 66 | 67 | 68 | 69 | 70 |
| Composition | Wt.-% | Wt.-% | Wt.-% | Wt.-% | Wt.-% | Wt.-% | Wt.-% | Wt.-% | Wt.-% | Wt.-% |
| SiO₂ | 72.4 | 71.2 | 68.9 | 68.9 | 70.7 | 70.7 | 69.7 | 75.00 | 75.00 | 72.6 |
| Li₂O | 10.3 | 10.1 | 9.8 | 9.8 | 10.0 | 10.0 | 9.9 | 10.7 | 10.7 | 10.2 |
| Na₂O | — | — | — | — | — | — | — | — | — | — |
| K₂O | 7.8 | 5.2 | 5.8 | 5.8 | 5.8 | 5.8 | 6.0 | 5.8 | 5.8 | 5.0 |
| Rb₂O | — | — | — | — | — | — | — | — | — | — |
| Cs₂O | — | — | — | — | — | — | — | — | — | — |
| MgO | — | — | — | — | — | — | — | — | — | — |
| CaO | — | — | — | — | — | — | — | — | — | 1.0 |
| SrO | — | — | — | — | — | — | — | — | — | — |
| ZnO | — | — | — | — | — | — | — | — | — | — |
| Al₂O₃ | 3.0 | 7.0 | 5.0 | 4.0 | 5.0 | 5.0 | 6.0 | 5.0 | 5.0 | 4.7 |
| Y₂O₃ | — | — | — | — | — | — | — | — | — | — |
| La₂O₃ | — | — | — | — | — | — | — | — | — | — |
| ZrO₂ | — | — | 4.0 | 4.0 | — | — | — | 1.0 | 1.0 | 1.0 |
| CeO₂ | — | — | — | — | — | — | — | — | — | — |
| P₂O₅ | 2.5 | 2.5 | 2.5 | 2.5 | 2.5 | 2.5 | 2.4 | 2.5 | 2.5 | 5.0 |
| V₂O₅ | — | — | — | — | — | — | — | — | — | — |
| Nb₂O₅ | — | — | — | — | — | 2.0 | 2.0 | — | — | — |
| Ta₂O₅ | — | — | — | — | 2.0 | — | — | — | — | — |
| Gd₂O₃ | 4.0 | 4.0 | 4.0 | 5.0 | 4.0 | 4.0 | 4.0 | — | — | — |
| Tb₄O₇ | — | — | — | — | — | — | — | — | — | — |
| Er₂O₃ | — | — | — | — | — | — | — | — | — | — |
| F | — | — | — | — | — | — | — | — | — | 0.5 |
| Σ | 100.0 | 100.0 | 100.0 | 100.0 | 100.0 | 100.0 | 100.0 | 100.0 | 100.0 | 100.0 |

| | Example | | | | | | | |
|---|---|---|---|---|---|---|---|---|
| | 71 | 72 | 73 | 74 | 75 | 76 | 77 | COMP |
| Composition | Wt.-% | Wt.-% | Wt.-% | Wt.-% | Wt.-% | Wt.-% | Wt.-% | Wt.-% |
| SiO₂ | 69.2 | 70.0 | 70.6 | 68.1 | 68.0 | 67.1 | 65.2 | 71.2 |
| Li₂O | 10.7 | 10.3 | 9.8 | 11.3 | 11.3 | 9.5 | 9.3 | 14.8 |
| Na₂O | — | — | — | — | — | — | — | — |
| K₂O | 5.5 | 5.2 | 5.2 | 5.7 | 5.7 | 5.6 | 5.4 | 4.0 |
| Rb₂O | — | — | — | — | — | — | — | — |
| Cs₂O | — | — | — | — | — | — | — | — |
| MgO | — | — | — | — | — | — | — | — |
| CaO | — | — | — | — | — | — | — | — |
| SrO | — | — | — | — | — | — | — | — |
| ZnO | — | — | — | — | — | 2.7 | 5.4 | — |
| Al₂O₃ | 4.0 | 4.0 | 4.0 | 4.0 | 4.0 | 4.9 | 4.7 | 3.3 |
| Y₂O₃ | — | — | — | 2.0 | 2.0 | — | — | — |
| La₂O₃ | — | — | — | — | — | — | — | — |
| ZrO₂ | 8.0 | 8.0 | 8.0 | 6.0 | 6.0 | 7.8 | 7.6 | 0.8 |
| CeO₂ | — | — | — | — | — | — | — | 1.9 |
| P₂O₅ | 2.6 | 2.5 | 2.4 | 2.9 | 3.0 | 2.4 | 2.4 | 3.2 |
| V₂O₅ | — | — | — | — | — | — | — | 0.1 |
| Nb₂O₅ | — | — | — | — | — | — | — | — |
| Ta₂O₅ | — | — | — | — | — | — | — | — |
| Gd₂O₃ | — | — | — | — | — | — | — | — |
| Tb₄O₇ | — | — | — | — | — | — | — | 0.5 |
| Er₂O₃ | — | — | — | — | — | — | — | 0.2 |
| F | — | — | — | — | — | — | — | — |
| Σ | 100.0 | 100.0 | 100.0 | 100.0 | 100.0 | 100.0 | 100.0 | 100.0 |

TABLE II

| Example | 1 | 2 | 3 | 4 | 5 | 6 | 7 | 8 | 9 | 10 |
|---|---|---|---|---|---|---|---|---|---|---|
| T_{g} [° C.] | 519 | 502 | 502 | 504 | 507 | 502 | 500 | 501 | 501 | 491 |
| Tₛ [° C.] | 1500 | 1550 | 1550 | 1550 | 1550 | 1550 | 1550 | 1550 | 1550 | 1550 |
| tₛ [min] | 60 | 60 | 60 | 60 | 60 | 60 | 60 | 60 | 60 | 60 |
| T₁ [° C.] | 500 | 520 | 520 | 520 | 520 | 520 | 520 | 500 | 500 | 510 |
| t₁ [min] | 10 | 10 | 10 | 10 | 10 | 10 | 10 | 10 | 10 | 40 |
| T₂ [° C.] | 870 | 880 | 840 | 850 | 850 | 830 | 830 | 810 | 810 | 840 |
| t₂ [min] | 10 | 10 | 60 | 10 | 10 | 10 | 10 | 10 | 10 | 10 |
| Main crystal | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ |
| phase | | | | | | | | | | |
| Phase port. | | | | | | | | | | |
| [wt.-%] | | | | | | | | | | |
| Average length | | | | | | | | | | |
| [μm] | | | | | | | | | | |
| Average aspect | | | | | | | | | | |
| ratio | | | | | | | | | | |
| Other crystal | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ |
| phases | | | Li₂SiO₃ | | | | | | LiAlSi₄O₁₀ | |
| | | | LiMgPO₄ | | | | | | | |
| Removal rate | | | | | | | | | | |
| [wt.-% · | | | | | | | | | | |
| min⁻¹] | | | | | | | | | | |
| L* | 92.58 | | | | | | | | | |
| a* | −0.81 | | | | | | | | | |
| b* | 6.20 | | | | | | | | | |
| CR | 92.58 | | | | | | | | | |
| K_{IC} [MPa | 1.68 ± | | | | | | | | | |
| m^{0.5}] | 0.17 | | | | | | | | | |
| σ_{B} [MPa] | 452 ± | | | | | | | | | |
| | 71 | | | | | | | | | |

| Example | 11 | 12 | 13 | 14 | 15 | 16 | 17 | 18 | 19 | 20 |
|---|---|---|---|---|---|---|---|---|---|---|
| T_{g} [° C.] | 495 | 491 | 522 | 522 | 502 | 482 | 513 | 505 | 506 | 505 |
| Tₛ [° C.] | 1600 | 1600 | 1500 | 1500 | 1500 | 1500 | 1500 | 1500 | 1500 | 1500 |
| tₛ [min] | 60 | 60 | 60 | 60 | 60 | 60 | 60 | 60 | 60 | 60 |
| T₁ [° C.] | 520 | 510 | 500 | 500 | 500 | 500 | 500 | 500 | 500 | 500 |
| t₁ [min] | 10 | 10 | 10 | 10 | 10 | 10 | 10 | 10 | 10 | 10 |
| T₂ [° C.] | 840 | 820 | 870 | 870 | 850 | 820 | 860 | 860 | 870 | 870 |
| t₂ [min] | 10 | 10 | 30 | 30 | 30 | 30 | 30 | 30 | 10 | 30 |
| Main crystal | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ |
| phase | | | | | | | | | | |
| Phase port. | 23.3 | 29.9 | | | | | | | | |
| [wt.-%] | | | | | | | | | | |
| Average length | 0.41 | 0.19 | | | | | | | | |
| [μm] | | | | | | | | | | |
| Average aspect | 2.44 | 1.97 | | | | | | | | |
| ratio | | | | | | | | | | |
| Other crystal | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ |
| phases | | | | Li₂SiO₃ | | | Li₂SiO₃ | Li₂SiO₃ | | |
| Removal rate | 15.95 | 15.41 | | | | | | | | |
| [wt.-% · | | | | | | | | | | |
| min⁻¹] | | | | | | | | | | |
| L* | | | 92.83 | | | | | | | |
| a* | | | −0.82 | | | | | | | |
| b* | | | 5.89 | | | | | | | |
| CR | | | 55.64 | | | | | | | |
| K_{IC} [MPa | | | | | | | | | | 1.98 ± |
| m^{0.5}] | | | | | | | | | | 0.21 |
| OB [MPσ_{B} [MPa]a] | | | | | | | | | | 497 ± |
| | | | | | | | | | | 44 |

| Example | 21 | 22 | 23 | 24 | 25 | 26 | 27 | 28 | 29 | 30 |
|---|---|---|---|---|---|---|---|---|---|---|
| T_{g} [° C.] | 503 | 502 | 496 | 499 | 503 | 505 | 504 | 506 | 514 | 504 |
| Tₛ [° C.] | 1500 | 1500 | 1500 | 1500 | 1500 | 1500 | 1500 | 1500 | 1550 | 1550 |
| tₛ [min] | 60 | 60 | 60 | 60 | 60 | 60 | 60 | 60 | 60 | 60 |
| T₁ [° C.] | 500 | 500 | 500 | 500 | 500 | 500 | 500 | 500 | 540 | 520 |
| t₁ [min] | 10 | 10 | 10 | 10 | 10 | 10 | 10 | 10 | 10 | 60 |
| T₂ [° C.] | 860 | 850 | 850 | 850 | 850 | 840 | 830 | 840 | 860 | 850 |
| t₂ [min] | 30 | 10 | 10 | 10 | 10 | 10 | 10 | 10 | 10 | 10 |
| Main crystal | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ |
| phase | | | | | | | | | | |
| Phase port. | | | | | | | | | | |
| [wt.-%] | | | | | | | | | | |
| Average length | | | | | | | | | | |
| [μm] | | | | | | | | | | |
| Average aspect | | | | | | | | | | |
| ratio | | | | | | | | | | |
| Other crystal | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ |
| phases | | | | | | | | Li₂SiO₃ | | |
| Removal rate | | | | | | | | | | |
| [wt.-% · | | | | | | | | | | |
| min⁻¹] | | | | | | | | | | |
| L* | | | 93.41 | 93.32 | 92.98 | 93.00 | 93.11 | | | 93.15 |
| a* | | | −0.68 | −0.77 | −0.72 | −0.82 | −0.78 | | | −0.81 |
| b* | | | 3.69 | 4.02 | 4.46 | 4.37 | 4.87 | | | 5.29 |
| CR | | | 45.72 | 48.26 | 43.28 | 42.50 | 43.76 | | | 47.08 |
| K_{IC} [MPa | | 2.24 ± | | 1.90 ± | | | 1.77 ± | | 1.89 ± | |
| m^{0.5}] | | 0.16 | | 0.06 | | | 0.12 | | 0.22 | |
| σ_{B} [MPa] | | 223 ± | 600 ± | | 349 ± | 459 ± | | | 309 ± | |
| | | 82 | 55 | | 127 | 80 | | | 40 | |
| Example | 31 | 32 | 33 | 34 | 35 | 36 | 37 | 38 | 39 | 40 |
| T_{g} [° C.] | 506 | 508 | 514 | 513 | 511 | 520 | 513 | 508 | 512 | 508 |
| Tₛ [° C.] | 1550 | 1500 | 1550 | 1550 | 1550 | 1500 | 1550 | 1550 | 1550 | 1500 |
| tₛ [min] | 60 | 60 | 60 | 60 | 60 | 60 | 60 | 60 | 60 | 60 |
| T₁ [° C.] | 560 | 500 | 540 | 520 | 560 | 500 | 540 | 520 | 560 | 500 |
| t₁ [min] | 10 | 10 | 10 | 60 | 10 | 10 | 10 | 60 | 10 | 10 |
| T₂ [° C.] | 850 | 840 | 850 | 850 | 840 | 850 | 850 | 850 | 840 | 840 |
| t₂ [min] | 10 | 10 | 10 | 10 | 10 | 10 | 10 | 10 | 10 | 10 |
| Main crystal | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ |
| phase | | | | | | | | | | |
| Phase port. | | | | | | | | | | |
| [wt.-%] | | | | | | | | | | |
| Average length | | | | | | | | | | |
| [μm] | | | | | | | | | | |
| Average aspect | | | | | | | | | | |
| ratio | | | | | | | | | | |
| Other crystal | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ |
| phases | | Li₂SiO₃ | | | | Li₂SiO₃ | | | | |
| Removal rate | | | | | | | | | | |
| [wt.-% · | | | | | | | | | | |
| min⁻¹] | | | | | | | | | | |
| L* | 93.17 | | | 93.27 | 93.44 | | | 93.44 | 93.45 | |
| a* | −0.85 | | | −0.93 | −0.80 | | | −0.92 | −0.81 | |
| b* | 4.47 | | | 4.66 | 3.81 | | | 4.47 | 3.72 | |
| CR | 46.09 | | | 48.50 | 48.26 | | | 50.21 | 49.63 | |
| K_{IC} [MPa | | | 1.62 ± | | | | 1.52 ± | | | |
| m^{0.5}] | | | 0.06 | | | | 0.09 | | | |
| σ_{B} [MPa] | | | 296 ± | | | | 223 ± | | | |
| | | | 100 | | | | 76 | | | |
| Example | 41 | 42 | 43 | 44 | 45 | 46 | 47 | 48 | 49 | 50 |
| T_{g} [° C.] | 507 | 502 | 499 | 503 | 500 | 500 | 498 | 492 | 497 | 500 |
| Tₛ [° C.] | 1500 | 1550 | 1500 | 1500 | 1500 | 1500 | 1500 | 1500 | 1500 | 1550 |
| tₛ [min] | 60 | 60 | 60 | 60 | 60 | 60 | 60 | 60 | 60 | 60 |
| T₁ [° C.] | 500 | 500 | 500 | 520 | 520 | 520 | 540 | 540 | 540 | 530 |
| t₁ [min] | 10 | 10 | 10 | 10 | 10 | 10 | 10 | 10 | 10 | 30 |
| T₂ [° C.] | 840 | 850 | 840 | 860 | 860 | 860 | 840 | 840 | 840 | 830 |
| t₂ [min] | 10 | 10 | 10 | 10 | 10 | 10 | 10 | 10 | 10 | 10 |
| Main crystal | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ |
| phase | | | | | | | | | | |
| Phase port. | | | | | | | | | | |
| [wt.-%] | | | | | | | | | | |
| Average length | | | | | | 0.43 | | | | |
| [μm] | | | | | | | | | | |
| Average aspect | | | | | | 2.03 | | | | |
| ratio | | | | | | | | | | |
| Other crystal | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ |
| phases | Li₂SiO₃ | | | | | | | | | |
| Removal rate | | | | | | | | | | |
| [wt.-% · | | | | | | | | | | |
| min⁻¹] | | | | | | | | | | |
| L* | | | | 92.86 | 92.90 | 92.95 | 92.99 | 92.84 | 92.57 | |
| a* | | | | −1.04 | −1.04 | −1.06 | −0.82 | −0.80 | −0.69 | |
| b* | | | | 6.52 | 6.43 | 6.29 | 5.40 | 6.23 | 6.09 | |
| CR | | | | 43.80 | 44.16 | 45.98 | 40.62 | 41.53 | 39.68 | |
| K_{IC} [MPa | | | | 1.88 ± | 1.87 ± | 1.97 ± | 1.94 ± | 1.87 ± | 2.12 ± | |
| m^{0.5}] | | | | 0.12 | 0.04 | 0.31 | 0.11 | 0.09 | 0.22 | |
| σ_{B} [MPa] | | | | 273 ± | 243 ± | 330 ± | 229 ± | 287 ± | 257 ± | |
| | | | | 63 | 46 | 94 | 74 | 66 | 66 | |
| Example | 51 | 52 | 53 | 54 | 55 | 56 | 57 | 58 | 59 | 60 |
| T_{g} [° C.] | 485 | 488 | 491 | 474 | 484 | 494 | 491 | 490 | 494 | 481 |
| Tₛ [° C.] | 1550 | 1550 | 1550 | 1550 | 1550 | 1550 | 1550 | 1550 | 1550 | 1550 |
| tₛ [min] | 60 | 60 | 60 | 60 | 60 | 60 | 60 | 60 | 60 | 60 |
| T₁ [° C.] | 530 | 540 | 540 | 540 | 550 | 540 | 520 | 520 | 550 | 520 |
| t₁ [min] | 30 | 10 | 10 | 10 | 40 | 10 | 10 | 10 | 40 | 10 |
| T₂ [° C.] | 830 | 840 | 830 | 820 | 820 | 850 | 840 | 830 | 830 | 840 |
| t₂ [min] | 10 | 10 | 10 | 10 | 10 | 10 | 10 | 10 | 10 | 10 |
| Main crystal | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ |
| phase | | | | | | | | | | |
| Phase port. | | | | | | | | | | |
| [wt.-%] | | | | | | | | | | |
| Average length | | | | | | | | | | |
| [μm] | | | | | | | | | | |
| Average aspect | | | | | | | | | | |
| ratio | | | | | | | | | | |
| Other crystal | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ |
| phases | | | | | | | | | | |
| Removal rate | | | | | | | | | | |
| [wt.-% · | | | | | | | | | | |
| min⁻¹] | | | | | | | | | | |
| L* | | 94.16 | 94.02 | | | | 94.41 | 94.44 | | 94.54 |
| a* | | −0.55 | −0.62 | | | | −0.55 | −0.41 | | −0.45 |
| b* | | 3.74 | 4.07 | | | | 3.52 | 2.47 | | 2.9 |
| CR | | 72.71 | 68.16 | | | | 74.94 | 71.23 | | 78.12 |
| K_{IC} [MPa | | | | | | | 1.78 ± | 1.87 ± | | |
| m^{0.5}] | | | | | | | 0.11 | 0.10 | | |
| σ_{B} [MPa] | | | | | | | 252 ± | 275 ± | | |
| | | | | | | | 88 | 101 | | |

| Example | 61 | 62 | 63 | 64 | 65 | 66 | 67 | 68 | 69 | 70 |
|---|---|---|---|---|---|---|---|---|---|---|
| T_{g} [° C.] | 480 | 495 | 504 | 496 | 488 | 484 | 489 | 481 | 481 | 475 |
| Tₛ [° C.] | 1550 | 1550 | 1550 | 1550 | 1550 | 1550 | 1550 | 1600 | 1600 | 1500 |
| tₛ [min] | 60 | 60 | 60 | 60 | 60 | 60 | 60 | 60 | 60 | 60 |
| T₁ [° C.] | 550 | 550 | 520 | 550 | 550 | 520 | 550 | 500 | 500 | 500 |
| t₁ [min] | 40 | 40 | 10 | 40 | 40 | 10 | 40 | 30 | 30 | 30 |
| T₂ [° C.] | 870 | 830 | 850 | 830 | 830 | 830 | 830 | 850 | 840 | 830 |
| t₂ [min] | 10 | 10 | 10 | 10 | 10 | 10 | 10 | 5 | 1 | 30 |
| Main crystal | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ |
| phase | | | | | | | | | | |
| Phase port. | 24.2 | 27.9 | | | | | | 35.3 | 33.4 | 22.9 |
| [wt.-%] | | | | | | | | | | |
| Average length | 0.51 | 0.20 | | | | | | 0.19 | | 0.34 |
| [μm] | | | | | | | | | | |
| Average aspect | 2.28 | 1.87 | | | | | | 2.26 | | 2.53 |
| ratio | | | | | | | | | | |
| Other crystal | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ | Li₃PO₄ |
| phases | | | | | | | | | | Cristobalite |
| | | | | | | | | | | Quartz |
| Removal rate | 13.89 | 33.99 | | | | | | | | |
| [wt.-% · | | | | | | | | | | |
| min⁻¹] | | | | | | | | | | |
| L* | | | 93.73 | | 94.02 | 94.18 | | | | |
| a* | | | −0.75 | | −0.51 | −0.53 | | | | |
| b* | | | 5.22 | | 4.05 | 4.08 | | | | |
| CR | | | 60.72 | | 69.66 | 73.7 | | | | |
| K_{IC} [MPa | | | | | | | | | | |
| m^{0.5}] | | | | | | | | | | |
| σ_{B} [MPa] | | | | | | | | | | |

| Example | 71 | 72 | 73 | 74 | 75 | 76 | 77 | COMP |
|---|---|---|---|---|---|---|---|---|
| T_{g} [° C.] | 504 | 513 | 508 | 496 | 508 | 508 | 501 | |
| Tₛ [° C.] | 1550 | 1550 | 1550 | 1550 | 1550 | 1550 | 1550 | |
| tₛ [min] | 60 | 60 | 60 | 60 | 60 | 60 | 60 | |
| T₁ [° C.] | 600 | 600 | 600 | 600 | 600 | 530 | 530 | |
| t₁ [min] | 30 | 30 | 30 | 30 | 30 | 10 | 10 | |
| T₂ [° C.] | 850 | 850 | 850 | 850 | 850 | 850 | 850 | |
| t₂ [min] | 30 | 30 | 30 | 30 | 30 | 30 | 30 | |
| Main crystal | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ | Li₂Si₂O₅ |
| phase | | | | | | | | |
| Phase port. | | | | | | | | |
| [wt.-%] | | | | | | | | |
| Average length | | | | | | | | |
| [μm] | | | | | | | | |
| Average aspect | | | | | | | | |
| ratio | | | | | | | | |
| Other crystal | Li₃PO₄ | Li₃PO₄ | Li₃PO₄ | Li₃PO₄ | Li₃PO₄ | Li₃PO₄ | Li₃PO₄ | Li₃PO₄ |
| phases | | | | | | Li₂SiO₃ | Li₂SiO₃ | |
| Removal rate | | | | | | | | 7.4 |
| [wt.-% · | | | | | | | | |
| min⁻¹] | | | | | | | | |
| L* | | | | | | | | |
| a* | | | | | | | | |
| b* | | | | | | | | |
| CR | | | | | | | | |
| K_{IC} [MPa | | | | | | | | |
| m^{0.5}] | | | | | | | | |
| σ_{B} [MPa] | | | | | | | | |

1. A lithium silicate glass ceramic comprising lithium disilicate as main crystal phase and comprising not more than 40 wt.-% of lithium disilicate crystals.

2. The glass ceramic according to claim 1, which comprises not more than 35 wt.-% of lithium disilicate crystals.

3. The glass ceramic according to claim 1, wherein the lithium disilicate crystals have an average length in the range from 10 to 1000 nm and an aspect ratio in the range from 1.0 to 5.0.

4. The glass ceramic according to claim 1, which comprises 62.0 to 80.0 wt.-% SiO₂.

5. The glass ceramic according to claim 1, which comprises 7.0 to 13.0 wt.-% Li₂O.

6. The glass ceramic according to claim 1, which comprises 2.0 to 12.0 wt.-% of further oxide of monovalent elements Me^{I}₂O, wherein Me 20 is selected from Na₂O, K₂O, Rb₂O, Cs₂O and mixtures thereof.

7. The glass ceramic according to claim 1, which comprises 3.0 to 12.0 wt.-% Al₂O₃.

8. The glass ceramic according to claim 1, which comprises 0.5 to 10.0 wt.-% P₂O₅.

9. The glass ceramic according to claim 1, which comprises at least one of the following components in the amounts indicated:

| Component | Wt.-% |
|---|---|
| SiO₂ | 62.0 to 75.0 |
| Li₂O | 7.0 to 12.0 |
| Me^{^{I}}₂O | 2.0 to 12.0, |
| Al₂O₃ | 3.0 to 12.0 |
| P₂O₅ | 0.5 to 5.0 |
| Me^{^{II}}O | 0 to 8.0 |
| Me^{^{III}}₂O₃ | 0 to 8.0 |
| Me^{^{IV}}O₂ | 0 to 12.0 |
| Me^{^{V}}₂O₅ | 0 to 9.0 |
| Me^{^{VI}}O₃ | 0 to 3.0 |
| Fluorine | 0 to 2.0, |

wherein
Me^{I}₂O is selected from Na₂O, K₂O, Rb₂O, Cs₂O, and mixtures thereof,
Me^{II}O is selected from MgO, Cao, Sro, ZnO and mixtures thereof,
Me^{III}₂O₃ is selected from B₂O₃, Y₂O₃, La₂O₃, Ga₂O₃, In₂O₃ and mixtures thereof,
Me^{IV}O₂ is selected from TiO₂, ZrO₂, GeO₂, SnO₂, CeO₂ and mixtures thereof,
Me^{V}₂O₅ is selected from V₂O₅, Nb₂O₅, Ta₂O₅ and mixtures thereof, and
Me^{VI}O₃ is selected from MoO₃, WO₃ and mixtures thereof.

10. The glass ceramic according to claim 1, wherein the molar ratio of SiO₂ to Li₂O is in the range of 2.5 to 4.0.

11. The glass ceramic according to claim 1, which comprises less than 12 wt.-% of secondary crystal phases, wherein the secondary crystal phases comprise one or more of lithium metasilicate crystals, lithium phosphate crystals, SiO₂ crystals, SiO₂ solid solutions, lithium aluminosilicate crystals, and ZrO₂ crystals.

12. The glass ceramic according to claim 1, which comprises less than 10 wt.-% of quartz crystals and/or quartz solid solutions.

13. A starting glass, which comprises the components of the glass ceramic according to claim 1, and comprises nuclei for the formation of lithium disilicate crystals.

14. The glass ceramic according to claim 1, wherein the glass ceramic is in the form of a powder, a granulate, a blank or a dental restoration.

15. The starting glass according to claim 13, wherein the starting glass is in the form of a powder, a granulate, a blank or a dental restoration.

16. A process for producing the glass ceramic according to claim 1, in which a starting glass is subjected to at least one heat treatment at a temperature of 400 to 1000° C. for a duration of 1 to 240 min.

17. The process according to claim 16, wherein
(a) the starting glass is subjected to a heat treatment at a temperature of 400 to 650° C. for a duration of 1 to 240 min to form starting glass with nuclei, and
(b) the starting glass with nuclei is subjected to a heat treatment at a temperature of 700 to 1000° C. for a duration of in particular 1 to 120 min to form the glass ceramic.

18. A process of using the glass ceramic according to claim 1 as dental material comprising producing a dental restoration or coating a dental restoration.

19. The method according to claim 18, wherein the glass ceramic is given the shape of the desired dental restoration by pressing or machining, wherein the shape comprises a bridge, inlay, onlay, veneer, abutment, partial crown, crown or facet.

20. A process for producing a dental restoration comprising a bridge, inlay, onlay, veneer, abutment, partial crown, crown or facet, in which the glass ceramic according to claim 1 is given the shape of the desired dental restoration by pressing or machining.
